(19) United States
(12) Patent Application Publication
(10) Pub. No.: US 2026/0272476 A1
(43) Pub. Date: Sep. 17, 2026

(54) Minimally Invasive Integrated Instrument Assembly for Spinal Surgery

(71) Applicant: Unintech GmbH, Düsseldorf (DE)

(72) Inventor: QINGUANG YOU, Düsseldorf (DE)

(73) Assignee: Unintech GmbH, Düsseldorf (DE)

(21) Appl. No.: 19/075,795

(22) Filed: Mar. 11, 2025

(51) Int. Cl. A61B 17/16; A61B 90/00

(52) U.S. Cl. CPC A61B 17/1615; A61B 17/1671; A61B 90/06; A61B 90/08; A61B 2090/062; A61B 2090/08021

(57) ABSTRACT

The present disclosure relates to a spinal surgical instrument assembly for spinal surgery, designed to provide an integrated solution for protection, operation, and visualization. In one embodiment, the spinal surgical instrument assembly includes a U-shaped tube, a T-shaped tube, and an endoscope. In another embodiment, the spinal surgical instrument assembly includes a U-shaped tube, a reamer, and an endoscope. Through an optimized integrated design, the instruments achieve the goals of soft tissue protection, quantitative operation, improved surgical precision, and simplified procedural steps. The reamer includes a hollow reamer channel, distal teeth, and scale markings on its inner wall, enabling precise bone removal under endoscopic visualization. The spinal surgical instrument assembly configurations include U-shaped tube/T-shaped tube/endoscope nesting and the U-shaped tube/T-shaped tube/endoscope nesting, accommodating different surgical scenarios.

### FIELD

The present disclosure relates to a surgical instrument assembly for spinal surgery, specifically an integrated assembly having a U-shaped tube, a T-shaped tube, a reamer, and an endoscope, belonging to the field of medical devices.

### BACKGROUND

In current spinal surgeries, key steps include foraminal shaping and bone removal. However, existing methods have the following limitations:
1. Surgical channels are poorly designed, failing to simultaneously meet soft tissue protection and instrument operational needs.
2. Lack of quantitative methods for bone removal leads to reliance on surgeon experience, reducing surgical precision.
3. Frequent instrument changes complicate the procedure, increasing risk and time costs.
4. The endoscope lacks effective integrated protection, risking lens contamination or restricted visibility.

To address these issues, the present disclosure provides a surgical instrument assembly that integrates protection, visualization, and precise operation.

### SUMMARY

In one aspect, the present disclosure related to a spinal surgical instrument assembly. In certain embodiment, the spinal surgical instrument assembly includes a U-shaped tube, a T-shaped tube, a reamer, and an endoscope. In one embodiment, an integrated nested configuration of the spinal surgical instrument assembly includes the U-shaped tube, the reamer, and the endoscope. In another embodiment, an integrated nested configuration of the spinal surgical instrument assembly includes the U-shaped tube, the T-shaped tube, and the endoscope.

In certain embodiments, the U-shaped tube includes a U-tube body, a U-tube handle, and a smooth stepped slope. The smooth stepped slope is located at a distal end of the U-shaped tube. A first end of the U-tube body is connected to the U-tube handle, and a second end the U-shaped tube forms the smooth stepped slope. The internal diameter of the U-shaped tube is greater than the external diameters of the T-shaped tube and the reamer.

In certain embodiments, the T-shaped tube includes a T-tube body, a T-tube handle, and a smooth curved slope. The smooth curved slope is located at its distal end. A first end of the T-tube body is connected to the T-tube handle, and a second end of the T-tube body forms the smooth curved slope. The internal diameter of the T-shaped tube is greater than the external diameter of an endoscope shaft.

In certain embodiments, the reamer includes a hollow reamer channel, a set of distal teeth at a distal end of the hollow reamer channel. Scale markings are shown on an inner wall of the hollow reamer channel. The reamer includes a reamer handle at a proximal end. The internal diameter of the hollow reamer channel is greater than the external diameter of the endoscope shaft.

In certain embodiments, the endoscope is positioned inside the T-shaped tube or reamer. The endoscope is used to visualize a surgical area and provides a working channel for instruments.

In certain embodiments, the proximal end of the U-shaped tube is U-shaped or V-shaped and achieves locking through a mating connection with the proximal end of the T-shaped tube.

In certain embodiments, the hollow reamer channel is made of metal, and the reamer handle is made of metal or plastic.

In certain embodiments, the scale markings on the inner wall of the hollow reamer channel are observable through the endoscope and indicate the depth of bone reaming.

In certain embodiments, the combination of the U-shaped tube and T-shaped tube provides a protective operational channel for the endoscope, isolating external muscles, nerves, and soft tissues from entering the tube.

In certain embodiments, an integrated nested configuration of the spinal surgical instrument assembly includes the U-shaped tube, T-shaped tube, and the endoscope. The distal end tongue plate of the T-shaped tube displaces internal tissues. The endoscope is used to visualize the surgical area within the T-shaped tube.

In certain embodiments, an integrated nested configuration of the spinal surgical instrument assembly includes the U-shaped tube, reamer, and endoscope. The hollow reamer channel performs reaming operations under endoscopic visualization. The scale markings on the inner wall quantify the depth of bone removal.

### BRIEF DESCRIPTION OF THE DRAWINGS

The accompanying drawings illustrate one or more embodiments of the present disclosure, and features and benefits thereof, and together with the written description, serve to explain the principles of the present disclosure. Wherever possible, the same reference numbers are used throughout the drawings to refer to the same or like elements of an embodiment, and wherein:

FIG. 1(A) is a schematic view of a spinal surgical instrument assembly having a U-shaped tube and a reamer to be used with an endoscope according to certain embodiments of the present disclosure;

FIG. 1(B) is a schematic view of a spinal surgical instrument assembly having a U-shaped tube and a U-shaped tub to be used with an endoscope according to certain embodiments of the present disclosure;

FIG. 2(A) is a front perspective view of the U-shaped tube according to certain embodiments of the present disclosure;

FIG. 2(B) is a rear perspective view of the U-shaped tube according to certain embodiments of the present disclosure;

FIG. 3(A) is a front perspective view of the T-shaped tube according to certain embodiments of the present disclosure;

FIG. 3(B) is a rear perspective view of the T-shaped tube according to certain embodiments of the present disclosure;

FIG. 4(A) is a front perspective view of the reamer according to certain embodiments of the present disclosure;

FIG. 4(B) is a rear perspective view of the reamer according to certain embodiments of the present disclosure;

FIG. 5(A) is a front perspective view of the spinal surgical instrument assembly having the U-shaped tube and the T-shaped tube to be used with the endoscope according to certain embodiments of the present disclosure;

FIG. 5(B) is a rear perspective view of the spinal surgical instrument assembly having the U-shaped tube and the T-shaped tube to be used with the endoscope according to certain embodiments of the present disclosure;

FIG. 6(A) is a front perspective view of the spinal surgical instrument assembly having the U-shaped tube and the reamer to be used with the endoscope according to certain embodiments of the present disclosure; and

FIG. 6(B) is a rear perspective view of the spinal surgical instrument assembly having the U-shaped tube and the reamer to be used with the endoscope according to certain embodiments of the present disclosure.

### DETAILED DESCRIPTION

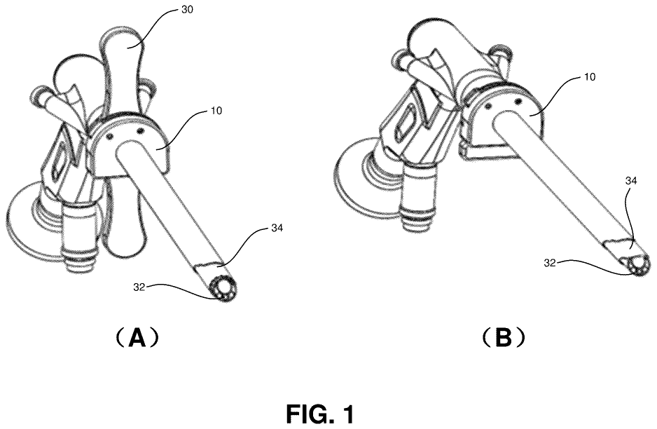

In one aspect, as shown in FIGS. 1-6, the present disclosure related to a spinal surgical instrument assembly 40. In certain embodiment, the spinal surgical instrument assembly 40 includes a U-shaped tube 10, a T-shaped tube 20, a reamer 30, and an endoscope. FIG. 1(A) illustrates an integrated nested configuration of the spinal surgical instrument assembly 40 having the U-shaped tube 10, reamer 30, and the endoscope. FIG. 1(B) illustrates an integrated nested configuration of the spinal surgical instrument assembly 40 having the U-shaped tube 10, T-shaped tube 20, and the endoscope.

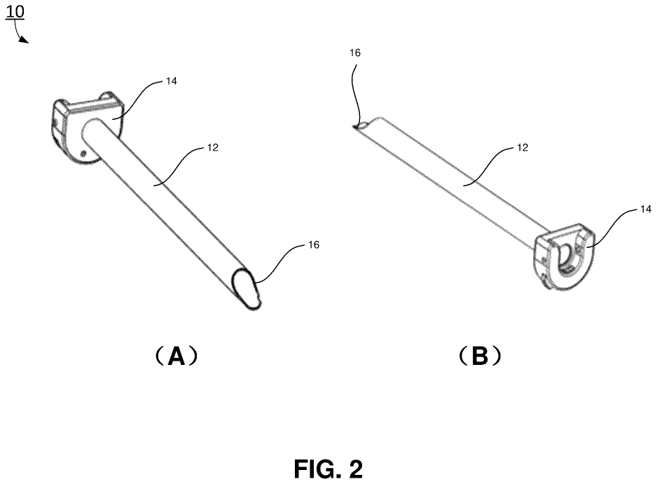

In certain embodiments, as shown in FIG. 2(A) and FIG. 2(B), the U-shaped tube 10 includes a U-tube body 12, a U-tube handle 14, and a smooth stepped slope 16. The smooth stepped slope 16 is located at a distal end of the U-shaped tube 10. A first end of the U-tube body 12 is connected to the U-tube handle 14, and a second end the U-shaped tube 10 forms the smooth stepped slope 16. The internal diameter of the U-shaped tube 10 is greater than the external diameters of the T-shaped tube 20 and the reamer 30.

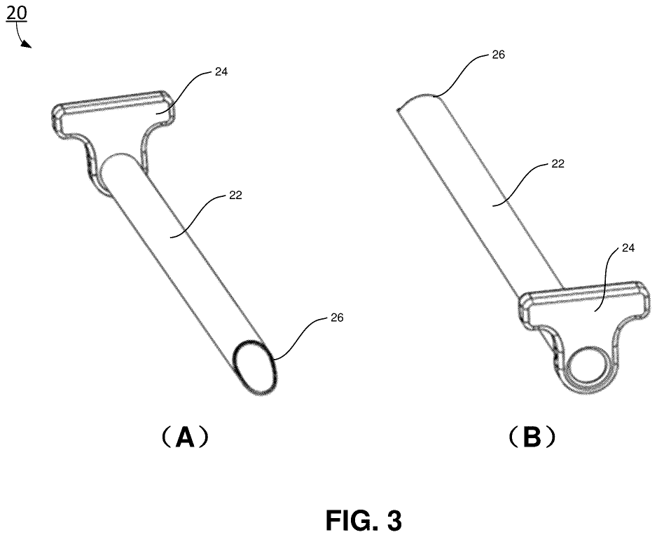

In certain embodiments, as shown in FIG. 3(A) and FIG. 3(B), the T-shaped tube 20 includes a T-tube body 22, a T-tube handle 24, and a smooth curved slope 26. The smooth curved slope 26 is located at its distal end. A first end of the T-tube body 22 is connected to the T-tube handle 24, and a second end of the T-tube body 22 forms the smooth curved slope 26. The internal diameter of the T-shaped tube 20 is greater than the external diameter of an endoscope shaft.

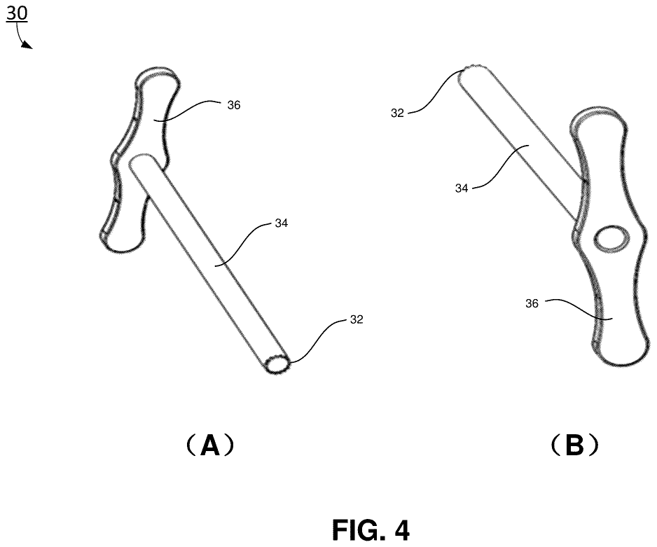

In certain embodiments, as shown in FIG. 4(A) and FIG. 4(B), the reamer 30 includes a hollow reamer channel 34, a set of distal teeth 32 at a distal end of the hollow reamer channel 34. Scale markings are shown on an inner wall of the hollow reamer channel 34. The reamer 30 includes a reamer handle 36 at a proximal end. The internal diameter of the hollow reamer channel 34 is greater than the external diameter of the endoscope shaft.

In certain embodiments, the endoscope is positioned inside the T-shaped tube 20 or reamer 30. The endoscope is used to visualize a surgical area and provides a working channel for instruments.

In certain embodiments, the proximal end of the U-shaped tube 10 is U-shaped or V-shaped and achieves locking through a mating connection with the proximal end of the T-shaped tube 20.

In certain embodiments, the hollow reamer channel 34 is made of metal, and the reamer handle 36 is made of metal or plastic.

In certain embodiments, the scale markings on the inner wall of the hollow reamer channel 34 are observable through the endoscope and indicate the depth of bone reaming.

In certain embodiments, the combination of the U-shaped tube 10 and T-shaped tube 20 provides a protective operational channel for the endoscope, isolating external muscles, nerves, and soft tissues from entering the tube.

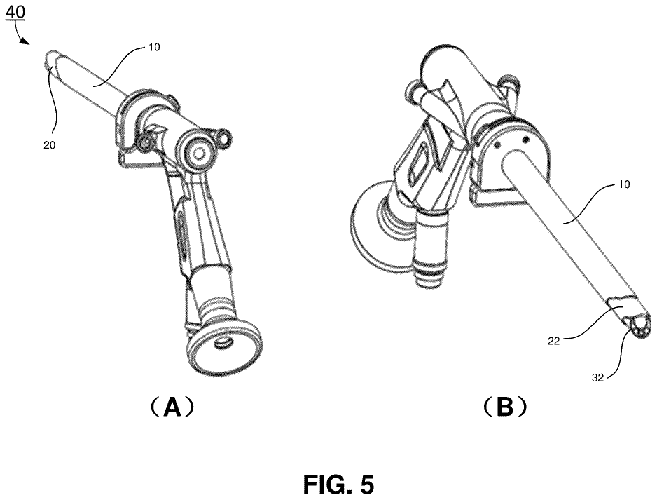

In certain embodiments, FIG. 5(A) and FIG. 5(B) illustrate an integrated nested configuration of the spinal surgical instrument assembly 40 having the U-shaped tube 10, T-shaped tube 20, and the endoscope. The distal end tongue plate of the T-shaped tube 20 displaces internal tissues. The endoscope is used to visualize the surgical area within the T-shaped tube 20.

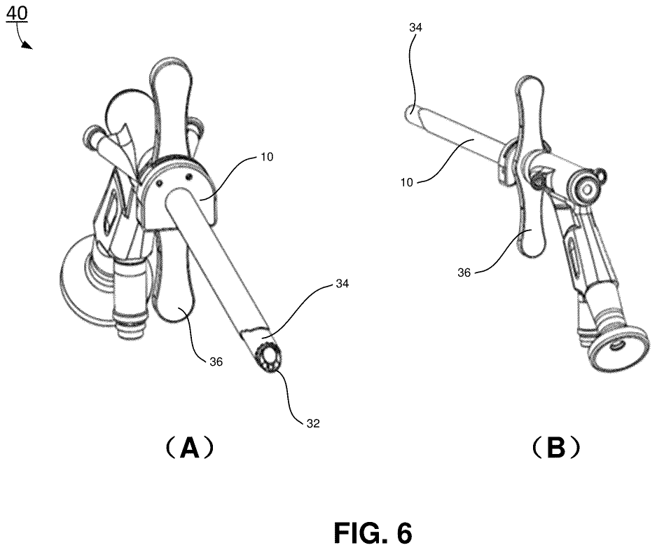

In certain embodiments, FIG. 6(A) and FIG. 6(B) illustrate an integrated nested configuration of the spinal surgical instrument assembly 40 having the U-shaped tube 10, reamer 30, and endoscope. The hollow reamer channel 34 performs reaming operations under endoscopic visualization. The scale markings on the inner wall quantify the depth of bone removal.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope. Accordingly, the scope of the present disclosure is defined by the appended claims rather than the foregoing description and the exemplary embodiments described therein.

What is claimed is:

1. A spinal surgical instrument assembly, comprising:
a U-shaped tube, wherein the U-shaped tube comprises a U-tube body, a U-tube handle, and a smooth stepped slope at a distal end, with a first end of the U-tube body fixed to the U-tube handle, and a second end of the U-tube body forming the smooth stepped slope, the internal diameter of the U-shaped tube being larger than the external diameters of the T-shaped tube and the reamer;
a T-shaped tube, wherein the T-shaped tube comprises a T-tube body, a T-tube handle, and a smooth curved slope at the distal end, with one end of the T-tube body fixed to the T-tube handle, and the other end having the smooth curved slope, the internal diameter of the T-shaped tube being larger than the external diameter of an endoscope shaft;
a reamer, wherein the reamer comprises a hollow reamer channel, with a plurality of distal teeth at its distal end, scale markings on the inner wall of the hollow reamer channel, and a reamer handle at a proximal end, the internal diameter of the hollow reamer channel being larger than the external diameter of the endoscope shaft; and
an endoscope, wherein the endoscope is positioned inside the T-shaped tube or reamer, the endoscope visualizes a surgical area and provides a working channel for instruments.

2. The spinal surgical instrument assembly according to claim 1, wherein the proximal end of the U-shaped tube is U-shaped or V-shaped and achieves locking through a mating connection with the proximal end of the T-shaped tube.

3. The spinal surgical instrument assembly according to claim 1, wherein the hollow reamer channel is made of metal, and the reamer handle is made of metal or plastic.

4. The spinal surgical instrument assembly according to claim 1, wherein the scale markings on the inner wall of the hollow reamer channel are observable through the endoscope and indicate the depth of bone reaming.

5. The spinal surgical instrument assembly according to claim 1, wherein the combination of the U-shaped tube and T-shaped tube provides a protective operational channel for the endoscope, isolating external muscles, nerves, and soft tissues from entering the tube.

6. The spinal surgical instrument assembly according to claim 1, wherein, in the integrated nested configuration of the U-shaped tube, T-shaped tube, and endoscope, the distal end tongue plate of the T-shaped tube displaces internal tissues, and the endoscope visualizes the surgical area within the T-shaped tube.

7. The spinal surgical instrument assembly according to claim 1, wherein, in the integrated nested configuration of the U-shaped tube, reamer, and endoscope, the hollow reamer channel performs reaming operations under endoscopic visualization, and the scale markings on the inner wall quantify the depth of bone removal.
